(19) United States
(12) Patent Application Publication
(10) Pub. No.: US 2009/0203986 A1
(43) Pub. Date: Aug. 13, 2009

(54) Medical data collection device

(76) Inventor: Sheldon Winnick, Walnut Creek, CA (US)

Correspondence Address: Lee Michael Pederson, 712 East Main Street, Sleepy Eye, MN, 56085, US

(21) Appl. No.: 12/321,480

(22) Filed: Jan. 21, 2009

Related U.S. Application Data
(60) Provisional application No. 61/011,790, filed on Jan. 22, 2008.
(60) Provisional application No. 61/130,918, filed on Jun. 3, 2008.
(60) Provisional application No. 61/189,334, filed on Aug. 17, 2008.

(51) Int. Cl. A61B 5/00; A61B 1/227; G06K 9/00

(52) U.S. Cl. 600/407; 382/128

(57) ABSTRACT

A medical data collection device is comprised of a head unit and a base unit. The head unit collects patient data and transmits the data to the base unit. The base unit captures, stores and processes the data A display on the base unit shows the captured data and allows the user to control the data.

### RELATED APPLICATIONS

This application claims priority from U.S. provisional patent application 61/011,790 entitled “Medical Data Collection Device” and filed on Jan. 22, 2008; U.S. provisional patent application 61/130,918 entitled “Medical Data Collection Device” and filed on Jun. 3, 2008; and U.S. provisional patent application 61/189,334 entitled “Medical Data Collection Device” and filed on Aug. 17, 2008.

### FIELD OF THE INVENTION

This invention relates to medical diagnostics, particularly to devices which gather and store data obtained from direct examination of a patient.

### BACKGROUND OF INVENTION

Medical professionals rely on a variety of data to inform their diagnoses and treatment plans. This data takes many forms including written notes and test results from a variety of instruments. It is desirable to integrate the collection, storage and dissemination of such data to improve effectiveness and efficiency.

### BRIEF DESCRIPTION OF THE INVENTION

A medical data collection device is comprised of a head unit and a base unit. The head unit collects patient data and transmits the data to the base unit. The base unit captures, stores and processes the data. A display on the base unit shows the captured data and allows the user to control the data. In a specific embodiment, the invention includes devices and methods for remotely diagnosing an ear infection using mostly off-the-shelf components.

### BRIEF DESCRIPTION OF THE DRAWINGS

FIG. 1 shows a schematic of the system.

FIG. 2 shows a schematic of the head unit.

FIG. 3 shows a schematic of the base unit.

FIG. 4 shows a schematic of the system incorporating a teaching tool.

FIG. 5 shows a schematic of an individual medical record.

FIG. 6 depicts a human figure as displayed by the system.

FIG. 7 depicts a schematic of an embodiment of the system for remotely diagnosing an ear infection using mostly off-the-shelf components.

### DETAILED DESCRIPTION OF PREFERRED EMBODIMENTS

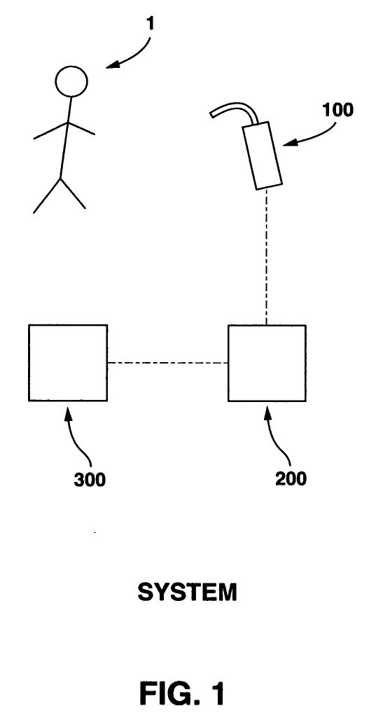

A medical data collection device is outlined in FIG. 1. A medical professional (not shown) uses a hand-held head unit 100 to collect data from a patient. Head unit 100 captures data including visual, audio, echogram, electrocardiogram, and lung function data. The data is processed into a digital signal and transmitted to base unit 200. Base unit 200 displays the data and stores the data in memory. Base unit 200 may also transmit the data to an outside computer or computer network 300.

Data transfer between the head unit and base unit is by means of radio signals, so that the medical professional has maximal freedom to manipulate the head unit. Data transfer between the base unit and the outside computer may be wireless or wired.

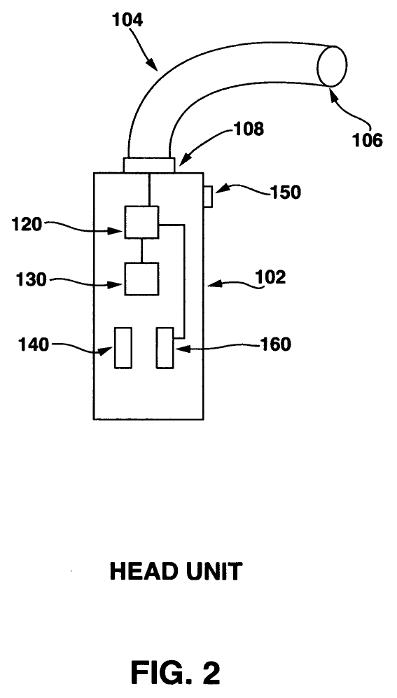

Referring to FIG. 2, the head unit 100 comprises a body 102. A sensor assembly 104 is attached externally to, or formed continually with, the body 102. Alternatively, the sensor assembly may be physically separate from the body and connected wirelessly. The sensor assembly 104 further comprises a sensor or sensors 106 capable of collecting patient data. An optional fitting 108 allows interchangeable sensor assemblies to be attached to the body 102. The head unit 100 may include one or more control triggers or switches 150. Such switches may be used to turn data capture functions on or off or to changes settings of the head unit, e.g., zooming a video image. A power supply 140 is included in the head unit 100.

Data collected from sensors 106 is transmitted to a processor 120. Typically the sensors will collect analog data which will be converted to digital form by the processor. The digital signal is sent to a first transmitter 130, which sends radio signals including the data to the base unit 200. In an alternative embodiment, the data may be sent as an analog signal to the body unit before analog to digital processing.

The head unit 100 may also comprise one or more displays 160. Such displays may include lights, video screens and audio speakers.

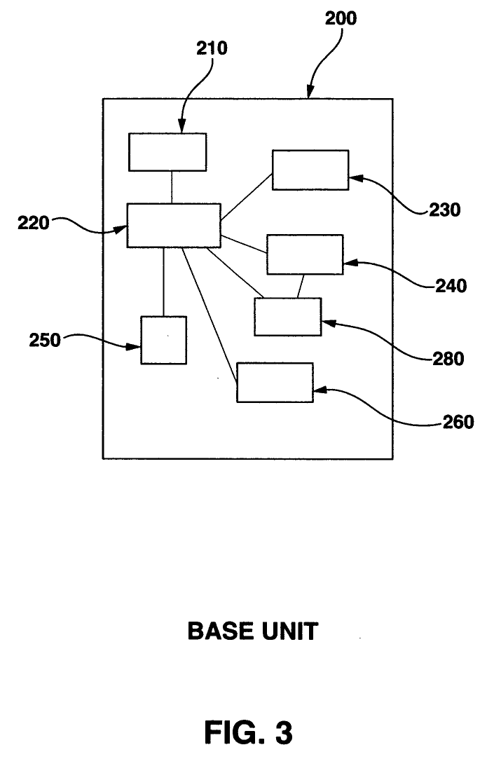

FIG. 2 describes the head unit only schematically. The actual physical implementation of a head unit is typically an ergonomic, light weight device that can easily be manipulated with one hand, leaving the user's second hand free. The head unit retains all the functionalities of the instruments it replaces, with the added benefit of digital data capture and storage. For example, when in otoscope mode a user can directly see the ear canal either via the display 160 or a conventional lens (not shown). Also for example, when in sonogram mode the user can directly view the data via a display 160 on the head unit or a display 250 on the base unit (shown in FIG. 3).

Head units with different sensors may differ greatly in physical form, while retaining the basic functionalities of a head unit as shown in FIG. 2. For example, an electrocardiogram head may comprises a set of pads for attaching to a patient's chest, where the sensor assembly comprises the pads and where the sensor assembly is connected to the body of the head unit either permanently or detachably. A head unit having a sensor assembly comprising a fiber optic camera for examining parts of the body where the sun don't shine will differ greatly in appearance from the electrocardiographic head. Head unit bodies may also take on a variety of forms depending on the type of patient data to be gathered.

Because the physician using the device can always view the patient data directly, the physician can select optimal conditions for capturing and storing the data digitally by using a trigger 150. This allows the physician to position the head unit for optimal data capture, providing the dual benefits of reducing the need to store redundant or suboptimal data and to record observations uniformly so that changes in a patient's condition can be more easily detected by comparisons with previously stored data.

Referring to FIG. 3, the base unit 200 comprises a receiver 210 which receives radio signals from first transmitter 130 in the head unit. Receiver 210 delivers the data to processor 220. Data may be stored to and retrieved from memory 240. Control panel 230 allows a user to control the collection and dissemination of data. The panel may also remotely control the head unit by means of radio signals. One or more displays 250 display live or retrieved data, machine function, and menus of functions for ease of use. Such displays may include lights, video screens, data screens and speakers. Control panel 230 and display 250 may be integrated, e.g., as a touch screen operated by fingers or stylus.

A prompter 280 may be incorporated into the base unit 200 or integrated into the outside computer or computer network 300. The prompter is a computer routine implemented by either hardware or software or a combination of hardware and software. The main function of the prompter is to prompt the medical professional to obtain specific data based upon the circumstances of the patient. The prompter relies upon patient specific data such as age, sex and weight, as well as flags noted in previous examinations. In this way the prompter serves certain functions of current paper medical charts. Using the patient specific data, the prompter activates a display to provide relevant data and recommendations to the medical professional. The prompter also uses patient data to suggest diagnosis and treatment options. A medication tracker is incorporated into the prompter to record treatment histories and check for pharmaceutical incompatibilities.

The system is capable of storing many types of primary patient data via using different head units and sensors. Microphones capture audio data. Cameras capture video and pictorial data. Fiber optic heads captured detailed video of eyes, ears, nose and throat. Thermometer head may capture body temperature. Peak flow meter sensors capture lung function data. Sono-echographic heads capture echogram data. Electrocariogram heads and stethoscope heads capture data on heart function. An oximeter can capture data on blood oxygen levels. Other types of data collection heads may be used with the system.

One or more wide-angle cameras may be included in the system. The wide angle cameras may be configured as part of the base unit or separately. If the system is deployed in an examination room, the camera or cameras may be permanently positioned to record all or part of an examination. The cameras may be operable by a switch or trigger on a head unit, to allow the physician to record selected aspects of the examination.

The system may include multiple head units or sensors functioning simultaneously. For example, audio and video heads may be used together, whether they are configured as multiple sensors in the same head unit or configured separately.

The sensors of the head unit may be configured in various ways without departing from the spirit of the invention. In one embodiment, many of the sensors are attachable and detachable from the housing 102 by the use of a fitting 108. In this embodiment, the fitting physically holds the sensor and also provides connections for transmitting data from the sensor to the processor 120. Some sensors such as camera, microphone, and video capture may be permanently integrated into the head unit. In a configuration with detachable sensors, a head unit package capable of recording a complete physical examination includes one head unit housing and a plurality of detachable sensors.

In another embodiment, a head unit may have all its sensors permanently incorporated. Such a head unit may or may not include all the functionalities necessary for recording a complete physical examination.

In another embodiment, a plurality of head units each includes one or more sensors. The sensors may or may not be detachable. In this configuration, a head unit package capable of recording a complete physical examination includes a plurality of head units. Additional detachable sensors may also be included.

When a particular sensor is enabled on a head unit, the head unit and sensor together may be referred to as a particular type of sensor head. For example, a head unit with an enabled EKG sensor may be referred to herein as an EKG head.

A primary use of the invention is to conduct and record general physical examinations. Typically such examinations will proceed in a specific order. A preferred order is head, eyes, ears, nose, throat, chest/lung, heart, abdomen, genitals, extremities, nervous system, nodes, skin and spine. The displays (either on the head unit and/or on the base unit) may be programmed to prompt the physician to record data. Especially in the case of follow-up examinations where previous symptoms, conditions or abnormalities have been noted, the system can remind the physician to record particular data.

In a preferred mode, a continuous audio stream is recorded during the general examination. Video may be recorded under the control of the user.

Information such as the patient's age, height, weight, blood pressure, reported symptoms, current medications and treatments may be recorded prior to or at the outset of a session. Such information may be recorded in a variety of ways including data uploads or the use of a data entry device such as a keyboard or voice recognition system. Patient information and data stored in the system can be used to substitute for paper records.

An examination may begin by starting the audio recording. The purpose of the examination may be stated for the record. While examining and palpating the head, wide angle video capture may be turned on to record the technique used. A head unit with a video sensor enabled is used to examine the eyes. The physician may record the eye examination with pictures and/or video. A video/otoscope head is then used to examine and record the condition of the ears, nose and throat. Stethoscope, peak flow meter and echogram heads are used for the chest, lungs and heart. Again, the physician records key data into the system. The examination continues with the physician selecting and using the appropriate heads and sensors.

Complete primary patient data is recorded by the end of such a session. Additionally, the physician may flag certain aspects of the session for specific follow-up. Such data recorded into the system may be used, for example, to prompt a patient to schedule a follow-up exam, and then prompt the physician to pay special attention to certain aspects of the patient's health during subsequent examinations.

An initial data collection session, where there is no existing patient data in memory, begins by an operator initiating the session. Patient identification information is recorded by the operator and stored in memory. To make an audio recording of the session, the operator turns on a microphone. The microphone may be located on the head unit, the base unit, or separately. The operator may enter audio data (e.g., “18 month old male patient has presented with symptoms of fever and crying.”) which is stored in memory. To examine the patient's ears, the operator selects a otoscopic head unit with fiber optic video sensors. The operator inserts sensor into a patients ear. Once the sensor is in place for optimal data collection, the operator activates a trigger and a view of inner ear is captured, displayed for operator on a display, transmitted to base unit and stored in memory. The examination continues with the operator selecting and employing sensors as appropriate.

A subsequent physical examination of a treated patient, where existing patient data is stored in memory, begins by the operator entering patient identification into the system. The system recalls the patient's data from memory and associates the new session with existing patient records. The prompter will activate a display, reminding the physician of pertinent details of the patient's previous condition and treatment. The prompter will further prompt the physician to collect specific new patient data based upon the previous condition and treatment. The physician will then collect and record new data, using the appropriate hand units and sensors.

The system can also be used for abbreviated tests, such as when only one or two observations are needed to monitor the patient's condition.

Analytic functions can be incorporated into the system. For example, measurements of skin lesions may be recorded by internal scales. The system may also include features to compare the state of a skin lesion over time.

Sensors with advanced analytic functions may also be used. Spectroscopic, temperature, chemical, and other measurements of various parts of the body that are highly predictive of particular conditions may be taken. For example, physical properties of the tongue may indicate certain diseases.

The system may include features to aid a medical professional in monitoring physical conditions over time. For example, the system may used stored data to prompt the medical professional to repeat earlier observations. For example, in surveying a patient's moles, the operator may, during an initial examination, record a flag regarding a picture of a mole on a patients left upper arm. In a subsequent visit, the system may prompt the medical professional to record a new view of the same mole.

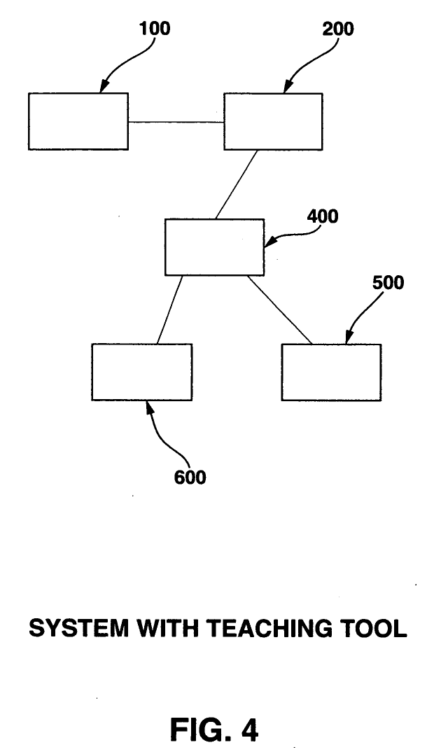

FIG. 4 shows a schematic of the system incorporating a teaching tool. When a teaching tool is incorporated with the system, the system includes head unit 100, base unit 200, teaching program 400, control unit 500, and display unit 600. Teaching program 400 uses patient data to create a visual or graphic display of the patient's physiological condition. Such patient data includes new patient data collected during an examination session, existing patient data collected from the patient during previous sessions, and existing data drawn from a universal database of normal and abnormal human physiological conditions. The physician uses the control unit 500 to manipulate the teaching program 400 to access new and existing data to create one or more graphic displays and display them on a display unit 600. The teaching program 400 is a computer routine implemented by either hardware or software or a combination of hardware and software. Teaching program 400 may be included in the base unit 200, or it may be included in an external computer or computer network 300.

The teaching tool is intended to facilitate communication and teaching between physician and patient by assisting the physician to describe the patient's condition and treatment options. For example, new patient data gathered with an echocardiographic head may show that a patient has an enlarged spleen. The physician can use the teaching tool to demonstrate to the patient their condition and the further tests needed to identify the cause of the enlargement. Once further tests are completed and the data is entered into the system, the physician can again use the teaching tool to demonstrate the cause and the treatment options.

The teaching tool is capable of generating many types of graphic displays including but not limited to patient data such as pictures or echocardiograms, three-dimensional views of the entire body, three dimensional views of body systems and organs, representations of cellular and molecular processes, statistical probabilities of the success of various treatment regimens, written word descriptions and symbolic representations. The physician may use control unit 500 to create such displays, such as by extracting video or activate the teaching program to execute particular routines. The physician may also use control unit 500 to edit such displays before the patient sees them. The physician may use an additional display (not shown) for such editing.

The many advantages of the invention include the preservation of unambiguous primary patient data. Audio recoding can largely replace written notes. All patient data can be maintained as an integrated digital file, providing instantaneous baseline data for aid in the diagnosis of new symptoms. Patient files can easily be transmitted to remote locations in case of emergency. Unlimited manipulation of the patient data for comparisons and consultations is possible. The system can be installed in a room, or configured as a portable device. Especially when incorporating a teaching tool, the device aids communication and teaching between doctor and patient.

The system is useful for providing an accurate record of a patient's condition, diagnosis, care and treatment. The main goal is to detect, record and monitor bio-information in a user-friendly and patient-friendly way. As a by-product of this approach, the system may improve doctor and patient relationships by enhancing the time spent in direct communication and dialoguing.

The system is also useful for practicing telemedicine. In one specific embodiment, the device is useful for remotely diagnosing ear infections.

Ear infections are a common childhood ailment, often treated by antibiotics. Typically, diagnosis of such an infection requires a parent or caregiver to make a special trip with the child for an in-person examination by a physician. This is often accompanied by waiting with other sick people in a waiting room. In addition to the inconvenience and possible exposure to other illnesses, the process also is prone to overuse of antibiotics, as the physician may be inclined to write a prescription as a precaution against possible infection. The doctor-patient interactions in such office visits is simple and straightforward, and may be substituted for by telemedicine devices and procedures described herein.

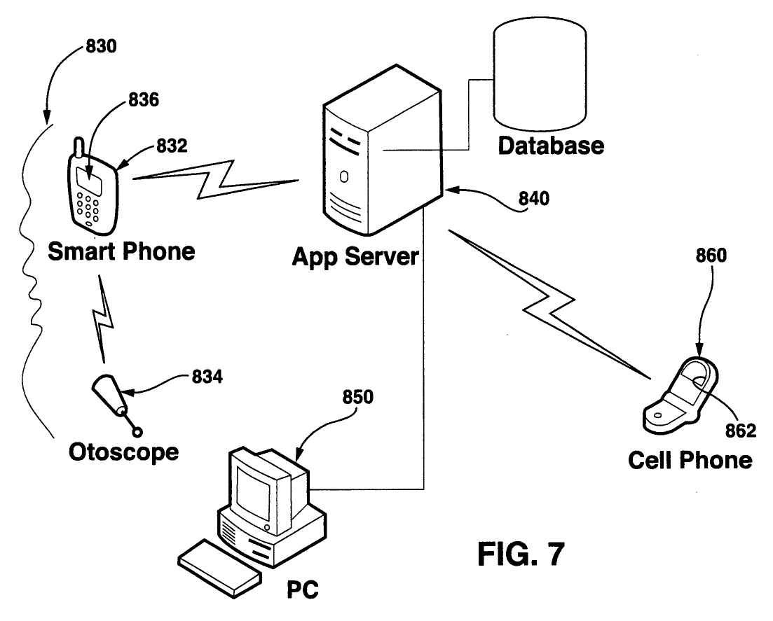

FIG. 7 depicts a schematic of an embodiment of the system for remotely diagnosing an ear infection using mostly off-the-shelf components. Most of the components in this system have been generally described in other figures, and their corresponding general designations may be included parenthetically. The system includes a head unit 830 (100 in FIG. 1). The head unit comprises a programmable wireless communication device (e.g., a cell phone) 832 and an otoscope sensor 834, which are preferably connected wirelessly, e.g., by a BlueTooth connection. Device 832 may have a display 836 (160) and communicates with a remote base unit 840 (200), which is in turn networked with other devices.

In practice, the system works by providing a parent or caregiver a head unit 830, along with training in how to operate it. The parent or caregiver uses the otoscope sensor 834 to capture images of the patient's inner ear, including the ear drum. Display 836 may be used to ensure that useful images are being captured. The images are transmitted to remote base unit 840 where they are viewed by the examining physician on a display 850. Alternatively, the images may be transmitted to a second wireless communication device (e.g., another cell phone) 860 having a display 862. In this manner, and in conjunction with audio communication, a physician may diagnose an ear infection almost no matter where the doctor and patient may be. If indicated, the physician may send a prescription to the patient (or the patient's pharmacy) without the need for an office visit.

Other aspects of the general system may be useful for such diagnosis. For example, the physician may use display 850 or device 860 to retrieve the medical records of the patient. Diagnostic routines embedded in the system may guide the physician in his or her diagnosis. Other sensors may be used by the patient or caregiver to capture addition data for the physician's diagnosis. Also, the diagnostic session data is stored in the patient's medical record for further use.

In one aspect, the invention is a device including a head unit plus a base unit. The device is used for collecting medical data from a patient. The device comprises a base unit and a head unit, where the base unit comprises a transceiver, a control panel, memory, a processor, and a first display. The head unit comprises a plurality of interchangeable sensors, and a transceiver for wireless communication with the base unit. The types of sensors may be selected from the group comprising still camera, video, sonographic, echographic, electrocardiographic, peak flow, stethoscopic and audio. The head unit further comprises a fitting for removably attaching a sensor. The head unit further comprises a switch for turning data capture on and off. The first display may be a touch screen display. The head unit may comprise a second display. The base unit may comprise a second transmitter. The device may further comprise a camera and/or a microphone separate from the head unit and the base unit. The separate camera may be a video camera. The base unit may comprise a second transmitter for communication with a computer network. The device may further comprise a prompter. The base unit may further comprise an input device. The head unit may further comprise an analog to digital processor.

In an alternative configuration, the device comprises a base unit and a plurality of head units, where the base unit comprises a transceiver, a control panel, memory, a processor, and a display; and each head unit comprises a sensor, and a transceiver for wireless communication with the base unit. The sensor type may be selected from the group comprising still camera, video, sonographic, echographic, electrocardiographic, peak flow, stethoscopic and audio. The head unit further may comprise a switch for turning data capture on and off. The first display may be a touch screen display. The head unit may comprise a second display. The base unit may comprise a second transmitter. The device may include a separate camera and or microphone. The camera may be a video camera. The base unit may comprise a second transmitter for communication with a computer network. The base unit may further comprise a prompter and/or an input device. Each head unit may further comprise an analog to digital processor.

In another aspect, the invention is a head unit device for collecting medical data comprising a body, a plurality of sensors, and a radio frequency transmitter. The sensors may be selected from the group comprising still camera, video, sonographic, echographic, electrocardiographic, peak flow, stethoscopic and audio. The device may further comprise a switch for turning data capture on and off. The device may further comprise an analog to digital processor. The device may further comprise a fitting for removably attaching a sensor. The device may further comprise a selector for enabling a specific sensor. The device may further comprise a lens. The device may further comprise a display. Such display may be a touch screen panel.

In an alternative configuration, the invention is a head unit device for collecting medical data comprising a body, a sensor, and a radio frequency transmitter. The type of sensor may be selected from the group comprising still camera, video, sonographic, echographic, electrocardiographic, peak flow, stethoscopic and audio. The device may further comprise a switch for turning data capture on and off. The device may further comprise an analog to digital processor, a lens and/or a display.

In another aspect, the invention is a base unit device for collecting medical data comprising a receiver for communicating with a head unit, a processor; a display, memory and a control panel. The display and control may be integrated into a touch screen panel. The device may further comprise a second transmitter for communicating with a computer network. The device may further comprise a prompter.

In another aspect, the invention is a method of collecting patient data during a physical examination session comprising selecting a first sensor on a wireless hand-held data collection head unit, capturing patient data using the first sensor, wirelessly transmitting the data from the head unit to a base unit and storing the data in digital form. The method may further comprise selecting a second sensor on the head unit, capturing patient data using the second sensor, wirelessly transmitting the data from the head unit to a base unit and storing the data in digital form. The selecting may be accomplished by removing the first sensor from the head unit and attaching the second sensor. The data may be, for example, a video image of the patient's throat or a peak flow meter reading.

In another aspect, the invention is a method of conducting a complete physical examination of a patient comprising recording an audio track of the entire examination; collecting images of the patients ears, nose and throat with a hand-held head unit with an otoscopic sensor, where the head unit transmits the images to a base unit for storage; collecting sounds of the patient's breathing and heartbeat with a hand-held head unit with a stethoscope sensor, where the head unit transmits the sounds to a base unit for storage; collecting lung function data with a hand-held head unit with a peak flow meter sensor, where the head unit transmits the data to a base unit for storage; collecting electrocardiogram data with a hand-held head unit with a peak electrocardiogram sensor, where the head unit transmits the data to a base unit for storage; and collecting internal organ data with a hand-held head unit with a sonogram sensor, where the head unit transmits the data to a base unit for storage.

In another aspect the invention is a device for enhancing communication between a physician and a patient comprising a head unit for collecting new patient data, a base unit for receiving the new data, a computer program for incorporating new patient data with existing patient data to create a graphic display of the patient's physiological condition, and a display unit. The graphic display may a three-dimensional view of the patient's body highlighting normal and existing body functions and conditions. The device may further comprise a control unit for previewing a graphic display before it is shown on the display unit.

In yet another aspect the invention comprises a method for enhancing communication between a physician and a patient. The method comprises collecting new patient data with a head unit, incorporating the new patient data with existing patient data, creating a graphic display of the patient's physiological condition, and displaying the graphic display to the patient. The graphic display may be a three-dimensional view of the patient's body highlighting normal and existing body functions and conditions. The method may further comprise manipulating the graphic display before displaying the graphic display to the patient.

Though communication between a head unit and the base unit is typically wireless, on occasion sensors may be connected to the base unit by wires. For example, a fixed microphone and camera in an examination room may be hard wired.

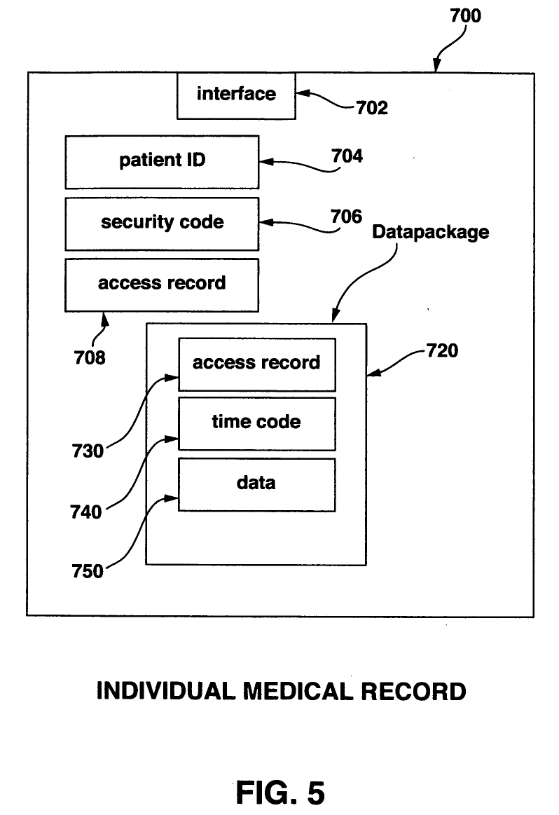

In another aspect the invention incorporates a medical data storage system. Data obtained through use of various head units, as well as all other medical data is digitally stored in individual patient medical records. Referring to FIG. 5, an individual medical record 700 is shown schematically. Each medical record includes an interface 702 to a data network. Each medical record includes a patient identifier 704. A security code 706 prevents unauthorized access to the record. An access record 708 keeps track of every attempt to access the medical record, including the person or account accessing the record, the time of access, and the information deposited, changed and/or requested.

Each medical record includes a plurality of data packets, such as data packet 720. In turn, each data packet 720 is identified by a field code 730 and a time code 740. The field code identifies the type of data in the data packet. Such data types may include data gathered from various head units, such as video images, still images, audio data, electrocardiogram data, electroencephalogram data, sonogram data, and the like. Other data types may include test results, DNA sequences, and diagnostic data from other sources. Still other data types include diagnoses and treatment records. The time code indicates when the data was obtained. The time codes and data codes are useful for retrieving specific data from a medical record, either by request from a human user or accession by a computer routine.

In the aspect of a medical data storage system, the invention includes a computer system coupled to a data network comprising an interface for accessing the data network and exchanging data signals therewith; a security mechanism to control access to the system; and a local persistent memory device for storing multiple individual medical records, where each individual medical record further comprises a patient identifier, a patient security code, an access record, and a plurality of data packages. Each data package may comprise a field code indicating the type of data stored in the data package; a time code indicating the time that the data was created; and patient medical data. The type of data stored in a data package may be selected from the group consisting of test results, images, DNA sequences, and primary data from diagnostic devices such as ekg, eeg, oximeter, ultrasound and video.

In yet another aspect the invention includes a medical diagnosis and display system. The system may be operated manually by a physician, who selects data packets from a medical record for direct human examination. Alternatively the system may be operated by one or more pattern recognition routines that access data packets and compare them against a database holding data describing both normal physiological conditions and clinical pathology states. Such a system may be operated by base unit 200 or other computers in the network 300.

In this aspect the invention includes a medical diagnosis and display system comprising a memory device for storing an individual medical record having a plurality of data packages; a database including data describing normal physiological conditions and clinical pathology states; a processor; a control panel; a pattern recognition routine for accessing data packages and comparing them against the database; and a display.

In another aspect the invention includes a method of performing a medical diagnosis comprising selecting a pattern recognition routine; accessing an individual medical record; accessing a database including data describing normal physiological conditions and clinical pathology states; comparing data in the individual medical record against data in the database; and observing the output of the routine.

In record capture, recording, reporting, and diagnosis modes, the system utilizes pattern recognition routines for where, when, and what a given pathological state exists. The system also tracks the clinical condition and therapeutic response compared at each point in time in a three-dimensional matrix of a body, with a point-to-point corresponding site of quantity and quality of lesion and/or physiologic state which can be captured and displayed easily for evaluation, comparison, storage, research and development of new diagnostic and therapeutic alternatives.

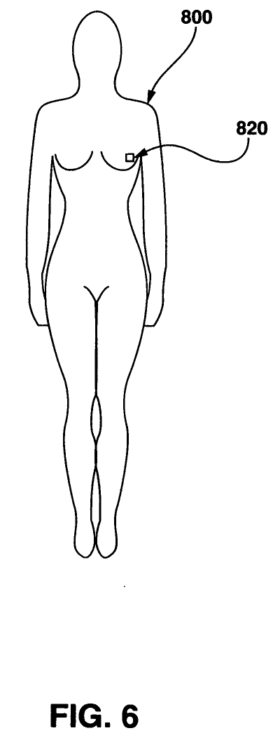

In another aspect, the invention comprises a system that integrates and displays medical data, as well as a method for integrating and displaying medical data. FIG. 6 depicts a female human FIG. 800 as displayed by the system. The system integrates data from various sources and, where possible, maps the data in three dimensions for diagnostic purposes as well as for enhancing doctor-patient communications. In practice, the display can be manipulated by a user to provide various perspectives. In this example, the FIG. 800 represents a breast cancer patient with a tumor 820.

All individual patient data since birth may be incorporated into the display system, whether obtained directly from the head unit 100 or from other sources. Such data includes information that readily allows depiction in three dimensions such as multiperspective still and video images, MRI scans, PET scans, X-rays, and the like. Information that is not readily displayed in three dimensions can also be displayed by the system. Such information includes lab tests, pathology reports, pathology images, treatment records, family histories, and the like.

When viewing the display of human FIG. 800, the user may zoom in on the area of the tumor 800. The display may be rotated to observe the tumor area from various perspectives. Overlays of systems (e.g., lymph) and tissues may be added to or removed from the display by the user. Two dimension cross-sections may be displayed. The user can also call all other patient data for display. Changes in physical and physiological conditions over time can also be displayed over time. For example, in the case of a breast cancer patient, a physician can view and display the condition over time. X-rays of breast examinations, stored in the system over a period of years, may be displayed chronologically. Records of past and current medical examinations, laboratory tests, pathology reports, treatment records, and the like can also be called up and displayed for review. The system can integrate and display all data for the entire human body, or any part thereof.

In another aspect the invention is a device for diagnosing ear infections where the physician and patient are physically separated, the device comprising a base unit and a head unit, where the base unit comprises a first transceiver, memory, and a processor. The head unit comprises an otoscope sensor, and a second transceiver for wireless communication with the base unit. The first transceiver of the head unit may be a first programmable communication device, e.g., a cell phone. The transceiver of the head unit and the otoscope sensor preferably communicate wirelessly, though wires may be used. The first programmable communication device may comprise a display programmed to allow viewing of images detected by the otoscope sensor. The device may further comprise an individual patient medical record. The device may further comprise a medical diagnosis and display system. The device may further comprise a pattern recognition routine for diagnosing ear infections. The device may further comprise a second programmable communication device for receiving images from the base unit.

In another aspect the invention is a method of diagnosing an ear infection, the method comprising imaging a patient's ear drum using an otoscope sensor; wirelessly transmitting the image of the ear drum from the sensor to a first programmable communication device; transmitting the image of the ear drum from the first programmable communication device to a remote base unit; and analyzing the image to determine a course of treatment. Such analyzing may include a computerized pattern recognition routine. The method may further comprise storing the images in an individual patient medical record. The method may further comprise displaying the image of the ear drum on a display on the first programmable communication device. The method may further comprise transmitting the image from the base unit to a second programmable communication device and displaying the image on the second programmable communication device.

In another aspect the invention includes an otoscope sensor head comprising a programmable communication device having a display; and an otoscope sensor, where the device and the sensor communicate wirelessly.

While preferred embodiments of the present invention have been described, those skilled in the art will recognize that other and further changes and modifications can be made without departing from the spirit of the invention, and all such changes and modifications should be understood to fall within the scope of the invention.

We claim:

1. A device for diagnosing ear infections where the physician and patient are physically separated, the device comprising a base unit and a head unit, the base unit comprising
a first transceiver,
memory, and
a processor, and
the head unit comprising
an otoscope sensor, and
a second transceiver for wireless communication with the base unit.

2. The device of claim 1 where the first transceiver of the head unit is a first programmable communication device.

3. The device of claim 2 where the transceiver of the head unit and the otoscope sensor communicate wirelessly.

4. The device of claim 2 where the first programmable communication device comprises a display programmed to allow viewing of images detected by the otoscope sensor.

5. The device of claim 1 further comprising an individual patient medical record.

6. The device of claim 1 further comprising a medical diagnosis and display system.

7. The device of claim 1 further comprising a pattern recognition routine for diagnosing ear infections.

8. The device of claim 1 further comprising a second programmable communication device for receiving images from the base unit.

9. A method of diagnosing an ear infection, the method comprising
imaging a patient's ear drum using an otoscope sensor;
wirelessly transmitting the image of the ear drum from the sensor to a first programmable communication device;
transmitting the image of the ear drum from the first programmable communication device to a remote base unit;
analyzing the image to determine a course of treatment.

10. The method of claim 9 where such analyzing includes a computerized pattern recognition routine.

11. The method of claim 9 further comprising storing the images in an individual patient medical record.

12. The method of claim 9 further comprising displaying the image of the ear drum on a display on the first programmable communication device.

13. The method of claim 9 further comprising transmitting the image from the base unit to a second programmable communication device and displaying the image on the second programmable communication device.

14. An otoscope sensor head comprising
a programmable communication device having a display; and
an otoscope sensor,
where the device and the sensor communicate wirelessly.
